(19) United States
(12) Patent Application Publication
(10) Pub. No.: US 2010/0233672 A1
(43) Pub. Date: Sep. 16, 2010

(54) GRAPE POLYPHENOLICS FOR PLATELET AND BACTERIAL CONTROL

(75) Inventor: Edward Shanbrom, Santa Ana, CA (US)

Correspondence Address: STEFAN KIRCHANSKI, VENABLE LLP 2049 CENTURY PARK EAST, 21ST FLOOR, LOS ANGELES, CA, 90067, US

(73) Assignee: Shanbrom Technologies, LLC, Ojai, CA (US)

(21) Appl. No.: 12/741,587

(22) PCT Filed: Nov. 6, 2008
(86) PCT No.: PCT/US08/82591

§ 371 Date: May 5, 2010

(30) Foreign Application Priority Data
Nov. 6, 2007 (US) 11935926

(51) Int. Cl. A01N 1/02; C07C 39/00

(52) U.S. Cl. 435/2; 568/717

(57) ABSTRACT

Special extracts of grape berries and Goji berries can be prepared by exposing fruit juices or preparations to an insoluble binding resin which is then extracted with soluble polyvinylpyrollidone. Grape and Goji extracts made in this way can be used to inhibit or control platelet aggregation. Grape extract has exceptional antibacterial properties and can be used to control oral bacteria and to control MRSA (Methicillin-resistant Staphylococcus aureus). The combination of control of platelet aggregation and antibacterial properties exhibited by the grape-extract allows it to be used to significantly extend the life of isolated platelets. When added to solutions of isolated platelets, the grape extract prevents bacterial growth and prevents deterioration of the platelets through activation. This treatment extends the usable life of platelet concentrates to at least ten days. In addition, polyphenols can be used as a medicament for modulation of platelet activity in vitro.

### CROSS-REFERENCE TO PRIOR APPLICATIONS

This application is based on and claims priority from U.S. patent application Ser. No. 11/935,926 filed on 6 Nov. 2007 which application is incorporated herein by reference to the extent permitted by applicable law.

### U.S. GOVERNMENT SUPPORT

Not Applicable

### BACKGROUND OF THE INVENTION

1. Area of the Art

The present invention concerns the area of medicinal uses of plant extracts and is particularly concerned with effects of grape polyphenolics on human blood cells both in vivo and in vitro.

2. Description of the Background Art

The circulatory system of mammals is protected by an amazingly complex coagulation system. Even a fairly large wound can be rapidly sealed before a life threatening loss of blood occurs. Yet the coagulation system is so elegantly controlled that the blood normally coagulates only at the site of an injury. The elegant control and specificity of the coagulation system is achieved through a combination of both cellular and soluble (humoral) components. There is usually no concern that the process of coagulation will become uncontrolled with blood clots spreading through out the circulatory system. However, there are situations where this fine tuned system does run amuck. Common vascular disease is one of these.

Vascular disease, particularly atherosclerosis, continues to be a major medical problem. A hallmark of this disease process is damage to the arteries in which the arteries are progressively occluded by “plaque.” This process is generally an inflammatory one and results in the growth of plaque between the inner endothelial lining and the smooth muscle wall of the artery. The plaque contains an infiltration of inflammatory cells and lipid; growth of the mass of plaque may gradually occlude the artery and impede the flow of blood (stenosis). Yet major medical problems do not invariably result directly from a narrowing due to the plaque. Instead the plaque becomes an area for inappropriate coagulation. The inflammatory process that forms the plaque results in localized damage to endothelial cells exposing molecules that stimulate platelet aggregation and clot formation.

Receptors on circulating platelets respond to the localized damage by binding to molecules exposed at the damaged area and by releasing a number of activating factors that cause other platelets to bind also and release still more activating factors. As a result a plug of platelets forms and fibrin fibers are synthesized from fibrinogen so that a full fledged blood clot forms at the site of the plaque. The clot may completely occlude the flow of blood. If this occurs in a coronary artery, an infarction or heart attack results. Some times pieces of the clot break off and lodge elsewhere in the circulatory system. If the clot lodges in the lung an embolism may result. If the clot finds its way to the brain, a stroke ensues. There are a variety of treatments aimed at reducing or avoiding the formation of arterial plaque. However, plaque forms slowly and silently over a long period of time, and treatments to reduce and reverse plaque formation may take an equally long period of time to be effective. In the meantime, the person with arterial disease is at significant risk for heart attack and stroke as a result of inappropriate clot formation.

Not unsurprisingly therapies aimed at reducing the tendency to form clots at the site of plaque are very important. Drugs such as heparin and warfarin which inhibit the soluble clotting factors are often used. Such treatments will generally not prevent platelets from aggregating in response to a plaque; however, they inhibit the platelets' ability to induce a full fledged clot. They also inhibit normal clot formation, for example at a wound, so that the level of these treatments must be carefully monitored lest a patient bleed to death from a minor wound. Further, such anticoagulants may predispose a patient to serious internal hemorrhages. An alternative approach is to interfere with the platelets' ability to aggregate at the site of a plaque. If platelet aggregation is inhibited, clots at the site of plaque can be prevented even though the remainder of the blood coagulation system is essentially intact. Drugs such as aspirin and clopidogrel (plavix) interfere with platelet aggregation by preventing synthesis of compounds that potentiate aggregation or by blocking receptors necessary for activation of the platelets. Because there is a number of different key platelet receptors involved in the process, it is generally possible to reduce platelet aggregation at plaque sites by interfering with only some, but not all, of the receptor so as to avoid dangerously compromising the body's ability to form effective clots at the site of a wound. Thus, treatments that lower the tendency for platelets to aggregate may be preferred over treatments that simply inhibit soluble clotting factors. Nevertheless, there are side effects that can limit the usefulness of anti-platelet aggregation drugs. Chronic use of aspirin can damage the stomach lining at the same time that its anticoagulant properties compromise the body's ability to prevent bleeding from such damage. Clopidogrel and similar drugs may produce other serious side effects and permanently (i.e., irreversibly) alter the properties of treated platelets. It will be appreciated that permanent alteration of treated platelets is advantageous in that inhibition of platelet aggregation continues between doses of the drugs; however, these same permanent changes can forestall or greatly complicate essential surgery. Therefore, non-permanent alternatives for preventing intravascular clots are highly desirable.

It is known that factors such as life style and diet can negatively or positively influence the outcome of vascular disease. Exercise and diet can significantly decrease the rate and extent of plaque formation. It also appears that diet can strongly influence the likelihood that existing plaque will result in serious blood clots. Therefore, it is not surprising that compounds in a number of foods mimic the anticoagulation and anti-platelet aggregation caused by drug treatments. Recent studies have demonstrated anti-thrombotic and anti-platelet properties in a variety of foods including strawberries (Blood Coagulation Fibrinolysis 16:501-9 [2005]), tomatoes (British Journal of Nutrition 90:1031-8 [2003]), mulberries (Platelets 17:555-64 [2006]), lichen extract (Ethnopharmacology 105:342-5 [2006]) and proanthocyanidin from grape seed (Thrombosis Research 115:115-21 [2005]) to name a few. Interestingly these studies showed that in many cases one variety of a fruit of vegetable would show the effect whereas another variety of the same fruit or vegetable would either have no effect or actually promote clot formation. The various foods were all effective in vitro and many were also effective in vivo. In some cases the foods could be shown to alter only platelets and in other cases the foods were shown to alter both platelets and coagulation.

An in vitro study compared the ability of alcohol, red wine and polyphenolic grape extract to alter the binding of platelets to fibrinogen and collagen at various shear rates (European Journal of Clinical Investigation 34:818-824 [2004]). These and other studies demonstrate an effect of various natural food components on platelets. Because these foods are widely consumed without negative consequences, it seems likely that a platelet treatment derived from natural foods would have few if any serious side effects. Unfortunately, none of these studies appear to provide a reproducible and easily quantifiable material for use in controlling platelet aggregation.

Although the above discussion has pointed out the health benefits of altering certain platelet properties, a functioning circulatory system absolutely depends on platelets. Without the microscopic “patches” mediated by platelets, one would quickly succumb to internal bleeding. Without platelets to mediate coagulation in cases of wounds one would most likely bleed to death from a simple cut. There are many medical conditions in which natural platelet production or function is impaired. Therefore, it should come as no surprise that transfusion of platelets is an extremely important medical procedure. Generally, platelets are prepared from whole donor blood by centrifugation or obtained directly from a donor through the process of plateletphoresis. In either case the donated platelets are valuable and in short supply. Because optimal platelet life requires storage of platelet concentrates at temperatures near or above room temperature, there is a significant danger of bacterial growth in such concentrates. That is, if any bacteria are present in the concentrate, they will rapidly multiply at elevated temperatures. In the case of whole blood used for transfusion the blood is stored under refrigeration so that bacterial growth is not a concern for at least thirty days.

Bacterial contamination of blood usually occurs because the skin surface that must be punctured to obtain blood is virtually impossible to completely sterilize. Thus, the most frequent bacterial contaminants of platelet concentrates are bacteria that commonly colonize the human skin. Because of the danger that older platelet concentrates may contain a large number of bacteria, United States Federal Food and Drug Administration rules generally limit the storage life of platelet concentrates to five days or fewer. This results in a significant waste of otherwise useable platelet concentrates because in the absence of bacteria, platelets can be stored for at least seven days or longer. The present inventor has proposed several treatments designed to extend platelet life, but up to now has not developed a completely successful treatment.

### SUMMARY OF THE INVENTION

Extracts of grape berries and Goji berries prepared by exposing grape or Goji fruit juices or preparations to an insoluble binding resin and then extracting the resin with soluble polyvinylpyrollidone have a number of novel uses. Grape and Goji extract can be used to inhibit or control platelet aggregation. Grape extract has exceptional antibacterial properties and can be used to control oral bacteria and to control MRSA (Methicillin-resistant Staphylococcus aureus) in a number of settings. The combination of platelet aggregation control and antibacterial properties exhibited by the grape-extract allows it to be used to significantly extend the life of isolated platelets. When added to solutions of isolated platelets, the grape extract prevents bacterial growth and prevents deterioration of the platelets through premature activation. This treatment extends the usable life of platelet concentrates to at least ten days.

In one embodiment grape-extract prepared according to the invention is added to isolated platelets to extend the life of the platelets. Before use of the platelets the grape extract can be removed by washing. In another embodiment platelet aggregation is inhibited by exposing platelets to Goji or grape extract. Such exposure can be achieved in vivo by injecting the extracts intravenously or by ingesting a sufficient quantity of the extracts. In another embodiment of the invention oral bacteria can be controlled either by administering an oral rinse containing grape extract or by consuming a confection containing grape-extract. In yet another embodiment MRSA is controlled by treating a site of MSRA infection or a site liable to infection with grape extract.

While most of the experiments were carried out with grape-extract, tests showed that polyphenolics from other fruits showed at least some activity. Therefore, the present invention includes the use of PVP-polyphenolics from other fruits to extend platelet life in vitro and modulate platelet activity in vivo.

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide methods for controlling platelet aggregation and safely extending the useful life of platelet concentrates.

The present inventor has long experimented with plant extracts produced by treating plant or fruit juices with binding materials such as crosslinked polyvinyl pyrollidone (PVP) and cholestyramine. Such extracts made from cranberry or blueberry juice are the subject of U.S. Pat. No. 6,093,401 (the “401” patent) which patent is incorporated herein by reference to the extent permitted by applicable law. While that patent was directed toward antibacterial properties of the resulting polyphenolic extracts, the present inventor has come to realize that extracts produced according to the method of that patent have other important properties and uses.

The inventor was intrigued by press reports concerning the Goji berry (Lycium barbarum) which has a variety of uses in traditional Chinese medicine including preventing aging and as well as treating a number of specific maladies. The Goji berry is known to be rich in antioxidants and important polyphenolic compounds. Recently Goji berries have been appearing in a variety of food products. Traditionally, Goji berries have been used to improve circulation, and one medical report indicates a dangerous potentiation of anticoagulant drugs (Ann Pharmacother. 2001 35(10):1199-201) by Goji berry juice. Based on these reports the inventor decided to test the effects of Goji extracts on platelets. Goji fruit (dried) was ground and treated with water to produce “juice” which was centrifuged to remove particulate material. The resulting liquid was then absorbed onto resin (cross-linked PVP or ion exchange resin Tulsion 412 (Thermax, Ltd.)) according to the method of the '401 patent. The bound material was then extracted by suspending 1 g of the resin in 10 ml of 10% (w/v) Kollidon PF12 (BASF) soluble PVP (10K to 15K MW). The suspended material was agitated for 4 hr at 37° C. The soluble PVP extracts the bounds material from the insoluble resin which is then removed from the solution by filtration or centrifugation. Produced in this manner the soluble solution is considered to be a 10% solution (1 g resin per 10 g solution) of the extract. The extracts are colored and can be measured spectrophotometrically directly or by using a reagent such as Folin's phenol reagent which responds to the polyphenolic components. Such measurements can be used to standardize the strength of extracts. As long as one starting batch of resin bound extract is used, all of the resulting soluble extracts should have reproducible strengths. When different starting batches of bound extract are use, spectrophotometric or phenolic standardization may be necessary to ensure comparable results.

Platelet aggregation can be measured by a variety of automated instruments using either optical or electrical impedance technologies. Thus, a sample of platelets can be submitted to a clinical laboratory for determination of platelet aggregation. The results are reported as “normal” or as some reduced percentage based on a normal reading. In vivo platelet aggregation is mediated by a number of different receptors on the surface of the platelets. It is usual to measure platelet aggregation in the presence of several different agonists as these measurements can provide additional details concerning the status of the platelets. For example, ADP (adenosine diphosphate) is a common agonist and normal platelets rapidly aggregate in the presence of ADP. The precise reason for ADP induced aggregation is not known although a purinoceptor known as P2Y₁ has been shown to be involved in ADP induced platelet aggregation. Collagen is also a frequently used agonist because collagen is exposed when vascular tissues (as well as other tissues) are damaged. Platelets have collagen receptors and quickly bind to the collagen and induce further platelet aggregation as well as blood coagulation. Epinephrine is a third platelet agonist. This “flight or fight” hormone is released under conditions of stress. Epinephrine results in rapid platelet aggregation partially mediated by alpha-adrenergic receptors on the platelets and partially mediated by fibrinogen receptors on the platelets. Finally, the antibiotic ristocetin is used to detect abnormalities in von Willebrand factor (vWf) because ristocetin potentiates binding of vWf to platelet receptors and results in platelet aggregation only in the presence of normal vWf in the plasma surrounding the platelets.

Goji-PVP was produced according to the above described method. Samples of platelet rich plasma (“PRP” prepared according to methods well known in the art) were exposed to 1% Goji-PVP extract (for example, 1 μl extract per 100 μl PRP) were sent with control samples (unexposed to Goji extract) to a clinical laboratory where they were exposed to standard amounts of platelet aggregation agonists. The resulting levels of aggregation were measured by an automated system and expressed as treatment results versus control. As shown in Table 1 the control PRP samples all responded normally to the agonists and the Goji extract treated samples reduced platelet aggregation.

TABLE 1

| Agonist | Control Aggregation | Goji Treated Aggregation |
|---|---|---|
| ADP | normal | 46% |
| Collagen | normal | 42% |
| Epinephrine | normal | 51% |
| Ristocetin | normal | 98% |

The observation that the control sample showed normal aggregation indicates that the PRP samples used were from donors with normal coagulation systems in their blood. The first three agonists were inhibited about 50% by the presence of the Goji extract. The Goji extract had virtually no effect on Ristocetin induced aggregation. Not shown is the PVP control which demonstrated that PVP alone had no measurable effect on platelet aggregation. This is not surprising since PVP has long been used as a safe plasma expander and would not be expected to alter platelet aggregation. What is interesting is the marked effect of relatively low concentration of the Goji extract on platelet aggregation. Many polyphenolics and other constituents of Goji and similar plant materials are known to be absorbed from the intestine and then excreted through the kidneys. While in circulation, these compounds would be expected to exert anti-platelet effects similar to those exhibited in vitro. Such effects may help to explain the correlation between diets rich in certain fruits and vegetables and vascular health (i.e., a lack of strokes and infarcts resulting from plaque related clots).

The inventor decided to see if other plant extracts produced by the same method showed similar properties. Because Goji had a reputation in traditional medicine, it was expected to be the most powerful plant extract in terms of platelet activity. An extract of commercial Concord grape juice (Vitis labrusca) was prepared according to the above method. The grape extract is generally more strongly colored than the Goji extract so attempts were made to standardize it to the Goji extract based on phenolic content rather than on color alone. Of course, because the compounds responsible for the Goji anti-platelet effect are not yet know, either approach to standardization might lead to artifact. In any case, Table 2 shows the grape extract was significantly more active than the Goji extract. It has not been possible to produce Goji extracts as efficacious as grape extracts. Thus, it seems likely that grape is inherently more active against platelets than the Goji.

TABLE 2

| Agonist | Control Aggregation | Grape Treated Aggregation |
|---|---|---|
| ADP | normal | 25% |
| Collagen | normal | 13% |
| Epinephrine | normal | 21% |
| Ristocetin | normal | 98% |

It appears that the difference between Goji and grape are qualitative (different active principles) as opposed to quantitative (amount of active principles). This can be deduced by looking at the grape-mediated inhibition of agonist-induced aggregation. With Goji the least inhibited agonist is epinephrine with the level of ADP inhibition being slightly higher and that of collagen inhibition being slightly greater than that of ADP. With grape the overall level of inhibition is significantly greater. Generally, the level of epinephrine inhibition is slightly greater than that of ADP while the inhibition of collagen-induced aggregation is much greater as compared to either epinephrine or ADP. Thus, the active ingredients in the grape extract are particularly effective in blocking aggregation mediated by collagen. This could be particularly important vascular damage caused by arterial plaque often exposes collagen so that it is likely that plaque induced clots are collagen mediated.

An experiment was then undertaken to determine whether the inhibition of aggregation is a permanent effect or takes place only in the presence of the grape extract. An aliquot of PRP was treated with grape-PVP as before, placed in a master tube and incubated at 21° C. Samples were withdrawn for reading by the clinical laboratory. However, some samples were “washed” prior to submission to the clinical laboratory. The samples were centrifuged to pellet the platelets, and then the pelleted platelets were resuspended in normal plasma (free of grape-PVP). As Table 3 indicated, this simple washing procedure essentially completely restored normal platelet aggregation. The very slight loss of aggregating ability may be a result of damage caused by the washing procedure or may represent the effects of a small residual level of grape-PVP.

TABLE 3

| | Control | Grape Treated | Washed Grape |
|---|---|---|---|
| Agonist | Aggregation | Aggregation | Treated |
| ADP | normal | 25% | 98% |
| Collagen | normal | 10% | 99% |
| Epinephrine | normal | 20% | 95% |
| Ristocetin | normal | 100% | 98% |

These results indicate that grape-PVP causes little if any permanent damage to the platelets. However, because the grape-PVP effectively blockades the platelets from activation, the inventor realized that grape-PVP represents a perfect means of preserving platelets during storage. Normally, storage of platelets presents two problems. As pointed out above, possible growth of bacteria in the platelet solutions limits the life of the concentrates to five days or fewer. However, as will be demonstrated below, grape-PVP is strongly antibacterial. Therefore, adding grape-PVP to platelet concentrates not only prevents platelet aggregation, it prevents growth of bacteria in the platelet solution. The second problem is that when a platelet becomes activated, it releases compounds (such as ADP) that cause other platelets to become activated and so on and so on. Thus, the longer one stores platelets, the more likely it is that an activation cascade will be initiated damaging or even destroying all the stored platelets. But grape-PVP reversibly blocks platelet activation and aggregation. This leads one to contemplate that platelet life will be prolonged in the presence of grape-PVP.

This hypothesis was tested by extending the experiment described in Table 3 beyond the first day. Each day additional samples of grape-PVP treated platelets were removed from the master tube, washed and sent to the clinical laboratory. The results are shown in Table 4. It can be observed that the platelet aggregation properties including the pattern of agonist effectiveness remains constant for at least 11 days. On day 14 it appears that both collagen and ADP results have climbed somewhat. This may be due to aging of the platelets.

TABLE 4

| | Day | Day | Day | Day | Day | Day | Day | Day | Day | Day | Day | Day | Day | Day |
|---|---|---|---|---|---|---|---|---|---|---|---|---|---|---|
| Agonist | 1 | 1W | 2 | 2W | 3 | 3W | 4 | 4W | 5 | 5W | 6 | 6W | 7 | 7W |
| ADP | 25% | 98% | 25% | 100% | 25% | 97% | 20% | 100% | 18% | 98% | 25% | 100% | 20% | 97% |
| Collagen | 10% | 99% | 15% | 100% | 10% | 98% | 10% | 99% | 15% | 98% | 14% | 100% | 10% | 98% |
| Epinephrine | 20% | 95% | 20% | 100% | 20% | 95% | 20% | 99% | 20% | 98% | 20% | 100% | 25% | 96% |
| Ristocetin | 100% | 98% | 99% | 99% | 99% | 100% | 98% | 100% | 100% | 98% | 97% | 100% | 98% | 98% |
| | Day | Day | Day | Day | Day | Day | Day | Day | Day | Day | Day | Day | Day | Day |
| Agonist | 8 | 8W | 9 | 9W | 10 | 10W | 11 | 11W | 12 | 12W | 13 | 13W | 14 | 14W |
| ADP | 20% | 100% | 25% | 99% | 20% | 95% | 25% | 100% | 30% | 98% | 30% | 100% | 32% | 100% |
| Collagen | 10% | 99% | 10% | 98% | 15% | 96% | 12% | 100% | 15% | 99% | 10% | 100% | 26% | 95% |
| Epinephrine | 20% | 95% | 20% | 99% | 18% | 98% | 20% | 99% | 25% | 96% | 20% | 95% | 25% | 96% |
| Ristocetin | 99% | 98% | 98% | 95% | 100% | 98% | 100% | 99% | 100% | 97% | 100% | 99% | 100% | 100% |

Significantly, the washed platelets come back to essentially normal values throughout the test. It is believed that random variations in the automated testing account for the slight “bounce” of the numbers. Importantly, the numbers for any particular agonist remain within a tight range throughout the test. An additional source of “noise” in the numbers may be the normalization of aggregation to fresh control platelets each day. Even were the control platelets drawn from the same donor each day, one would necessarily expect daily variation in platelets as a person's physiology changes from day to day. What is significant is that the addition of grape-PVP to platelet concentrates allows the concentrates to be safely stored for at least ten days—double the present five day life for platelet concentrates.

Grape-PVP is strongly antibacterial. This is illustrated by the profound effect that grape-PVP has on human bucal cavity bacteria. The bacterial flora of the mouth is complex, and a human mouth may contain hundreds of species of bacteria. Furthermore, oral bacteria have been detected in arterial plaque, and some theorize that these bacteria play a role in the etiology of plaque formation. It is fairly simple to demonstrate the extent of bacterial colonization of the bucal cavity. For this experiment either the teeth at the gum line or the surface of the tongue was swabbed with a sterile applicator or the mouth was rinsed and the swab samples or the rinse samples were spread on nutrient agar plates and incubated at 35° C. overnight. Following incubation the number of bacterial colonies was counted. Each colony represents a single bacterium from the swab or rinse. Swabbing was done either before of immediately after a 15 ml 30 second rinse with either sterile saline (0.9% w/v) or sterile 5% grape-PVP. The results are shown below in Table 5.

TABLE 5

| Source | Colony Count |
|---|---|
| Tooth/Gum Swab | 368 |
| Tongue Swab | Too Numerous to Count |
| Saline Rinse | 480 |
| Post Saline Tooth/Gum Swab | 353 |
| Post Saline Tongue Swab | Too Numerous to Count |
| Grape-PVP Rinse | 42 |
| Post G-P Tooth/Gum Swab | 31 |
| Post G-P Tongue Swab | 120 |

The sterile saline rinse had essentially no effect on the bacterial status of the tooth/gum surface. This is not surprising because the bacteria on the tooth surface are attached by biofilms that are extremely difficult to disrupt. Because the concentration of bacteria on the tongue surface is so high, it is impossible to determine whether the saline rinse had any appreciable effect. However, the number of bacteria in the rinse solution was relatively small suggesting that the bacteria on the tongue surface are too strongly attached to be removed by a saline rinse. However, the grape-PVP rinse was very effective in reducing the number of bacteria even on the tongue surface. Grape-PVP is useful as a simple oral rinse for reducing the number of oral bacteria. A more sustained result can be achieved by compounding the grape-PVP as a chewable or suckable substrate. For example, grape-PVP can be added to solid or chewable confections produced according to well-known recipes. For these purposes it is advantageous to use non-nutritive sweeteners although sugars are not absolutely counter-indicated. Clearly the grape-PVP contains potent bacteriostatic and/or bactericidal component. These components are responsible for preventing the growth of bacteria in treated platelet concentrates.

Of perhaps even greater significance in terms of the antibacterial properties of grape-PVP is the discovery that the material shows significant activity against MRSA (Methicillin-resistant Staphylococcus aureus). These bacteria first appeared in hospitals and rapidly became a common and hard to treat nosocomial infections. Surveys have found that on the average patients with MRSA infections have hospital stays that are three times longer and three times more expensive than patients without such an infection. Furthermore, the infected patients are five times more likely to die in the hospital. Most likely these statistics are partially caused by the fact that weaker and immunocompromised patients are more likely to develop MRSA infections. Now, however, the infections are appearing within the community where they strike healthy individuals and have proven to be quite contagious. One of the few treatments for MRSA involves the use of glycopeptides antibiotics like vancomycin. However, there is a significant danger that the infections will become resistant to even vancomycin.

Grape-PVP was tested against MRSA by preparing a 10% solution (1 g grape-insoluble resin in 10% Kollidon, 10 g total) as explained above. Following centrifugation at 2,000×g to remove the insoluble resin, the solution was adjusted to pH 5.0 and sterilized by filtration through a 0.2 μm membrane filter. Twelve two-fold serial dilutions were made of the grape-PVP using pH 5.0 trypticase soy broth. The dilutions were prepared in a 96 well microtiter plate, and the final volume in each well was 0.1 ml. Each well was inoculated with 1×1-5 MRSA organisms, and the plates were incubated overnight at 35° C. The MIC (minimal inhibitory concentration) was obtained by determining the lowest titer at which no bacterial growth was observed The MIC was found to be 1:32. The MBC (minimum bactericidal concentration) was determined by taking 0.02 ml of each well showing no growth in the MIC test and adding a 0.02 ml portion to fresh 0.1 ml trypticase soy agar well. This was incubated at 35° C. for 48 hr so that any inhibited (but not killed) cells could grow out. MCB represents the lowest titer where no cells survived. The MCB determined was to be 1:32. This means that there were no surviving bacterial cells in any of the no growth MIC wells. The MCB appears to be below the level of grape-PVP used in the in vitro tests and intended for the in vivo tests. The value of adding grape-PVP to MRSA treatments should not be underestimated.

Grape-PVP can be used in several different fashions to control MRSA. In a hospital MRSA infections often occur at sites of puncture wounds, catheters, etc. Such infections can be controlled by swabbing the area of the skin prior to introducing catheters, etc. Sites of wounds or MRSA infections can be treated directly with grape-PVP. Catheters and other medical instruments can be coated with grape-PVP.

As would be expected, the grape-PVP shows significant activity over a wide variety of bacteria other than MRSA. In addition, grape-PVP is generally more effective than cranberry-PVP extract which has been extensively studied by the present inventor. For the following test PVP extracts of both grape and cranberry measured MBC according to the methods described above for MRSA. A variety of bacterial strains were obtained from the American Type Culture Collection (ATCC): E. coli (Escherichia coil), K. pneumoniae (Klebsiella pneumoniae), Sa. enteritidis (Salmonella enteritidis), Sr. marcescens (Serratia marcescens), Ci. fruendii (Citrobacter freundii), Ps. aeruginosa (Pseudomonas aeruginosa), En. cloacae (Enterobacter cloacae) and En. faecalis (Enterobacter faecalis). Because fruit juices like cranberry are generally acidic, there has been a theory that antibacterial properties of the juice are due primarily to pH. PVP extracts are not acidic because the PVP does not bind fruit acids. Nevertheless, the results (shown in Table 6) were measure both at pH 7.0 and pH 5.0. It was anticipated that the material would be more effective at the acid pH; this proved not to true in all cases.

MBC

| | |
|---|---|
| Cran-PVP | Grape-PVP |

| | pH 7 | pH 5 | pH 7 | pH 5 |
|---|---|---|---|---|
| E coli | 1:8 | 1:8 | n/a | 1:32 |
| K. pneumoniae | 1:16 | 1:2 | 1:16 | 1:8 |
| Sa. enteritidis | n/a | n/a | n/a | 1:16 |
| Sr. marcescens | n/a | n/a | 1:2 | 1:32 |
| Ci. freundii | n/a | 1:8 | 1:128 | 1:128 |
| Ps. aeruginosa | n/a | 1:4 | 1:8 | 1:32 |
| En. cloacae | n/a | n/a | 1:16 | 1:4 |
| En. faecalis | n/a | n/a | n/a | 1:2 |

Clearly grape-PVP shows an effect against a wide range of bacteria. Where parallel results are available, the grape-PVP is as effective as and usually significantly more effective than the cranberry product. This is particularly true with Citrobacter freundii where the grape-PVP is dramatically more effective. Citrobacter causes a rare but deadly septicemia. Considering the dramatic sensitivity of Citrobacter to grape-PVP one would expect intravenous grape-PVP to be particularly effective as a treatment for Citrobacter septicemia.

The experiments described above are all in vitro tests. For preservation of platelet concentrates the in vitro test is in fact the actual test. However, for modulation of platelet aggregation in vivo it will be necessary to maintain sufficiently high plasma levels of the grape-PVP components. Animal experiments have been undertaken to demonstrate the safety and efficacy of injected grape-PVP. It is already known that PVP itself can be safely administered intravenously since the compound has a long history of use as a plasma volume expander. It is believed that the grape compounds extracted by the insoluble PVP will be harmless because they are commonly found in foods. Preliminary experiments in which PVP plant extracts were injected into animals demonstrated no obvious toxicity. The animals showed no overt response, and the colored components of the extracts were excreted in the urine and stools. Grape-PVP was tested in rabbits by administering 5 ml. 10 ml or 20 ml of a 10% grape-PVP prepared as explained above. Following administration of the grape-PVP blood was drawn from the animals for evaluation of platelet aggregation and other platelet functions. Based on the average blood volume of rabbits the 20 ml administration should result in a plasma concentration of grape-PVP at least as high as that in the in vitro experiments.

A control animal receiving 5 ml of PVP alone showed normal vital signs and blood measurements as would be expected. It was found that when rabbits were treated with 5 ml of grape-PVP all vital signs as well as blood measurements (CBC and white cell differential as well as blood chemistry) were unaffected by the treatment. The only measurable effect of the grape-PVP injection was on platelet aggregation at 15 min and 60 min after injection. By 24 hr the platelet response was essentially normal. These results are shown below in Tables 6 and 7. It would appear that the affect is beginning to subside by 60 min. By 24 hr following injection platelet aggregation had returned to normal.

TABLE 6
Rabbit #1 with 5 ml grape-PVP

| | | 15 min after | 60 min after |
|---|---|---|---|
| Agonist | Control | injection | injection |
| ADP | normal | 72% | 75% |
| Collagen | normal | 81% | 80% |
| Epinephrine | normal | 77% | 75% |
| Ristocetin | normal | 99% | 100% |

TABLE 7
Rabbit #2 with 5 ml grape-PVP

| | | 15 min after | 60 min after |
|---|---|---|---|
| Agonist | Control | injection | injection |
| ADP | normal | 75% | 75% |
| Collagen | normal | 79% | 77% |
| Epinephrine | normal | 83% | 79% |
| Ristocetin | normal | 99% | 100% |

Treatment of rabbits with 10 ml of PVP had no discernable effects on blood count, blood chemistry or platelet aggregation. Treatment of the rabbits with 10 ml of the grape-PVP showed similar results in that all vital signs were normal. With the larger dose of grape-PVP the effects on platelet aggregation were much more profound as well as much longer lasting. With the 5 ml grape-PVP treatment the platelet aggregation returned essentially to normal within 24 hours. With the injection of a greater concentration of grape-PVP platelet aggregation was still somewhat suppressed at 48 hours; at 72 hours after injection, the aggregation was essentially normal. This is extremely interesting because the in vivo experiments showed that platelet aggregation returned to normal upon washing the platelets. Therefore, the 10 ml grape-PVP experiments (shown below in Tables 8 and 9) imply that the active principle in grape-PVP is only relatively slowly cleared from the circulation. This suggests that the antibacterial properties of injected grape-PVP will be relatively long lasting.

TABLE 8
Rabbit #1 with 10 ml grape-PVP (times are after injection)

| Agonist | Control | 15 min | 60 min | 24 hr | 48 hr | 72 hr |
|---|---|---|---|---|---|---|
| ADP | normal | 32% | 30% | 35% | 88% | 99% |
| Collagen | normal | 44% | 40% | 46% | 90% | 99% |
| Epinephrine | normal | 30% | 28% | 32% | 90% | 100% |
| Ristocetin | normal | 100% | 100% | 99% | 99% | 100% |

TABLE 9
Rabbit #2 with 10 ml grape-PVP

| Agonist | Control | 15 min | 60 min | 24 hr | 48 hr | 72 hr |
|---|---|---|---|---|---|---|
| ADP | normal | 26% | 21% | 26% | 77% | 100% |
| Collagen | normal | 25% | 21% | 25% | 81% | 99% |
| Epinephrine | normal | 20% | 17% | 23% | 76% | 99% |
| Ristocetin | normal | 100% | 99% | 99% | 100% | 98% |

These results demonstrate the expected effect on platelet aggregation. The results on platelets were more long lasting than the effects of the 5 ml treatment. As might be expected, administration of 20 ml grape-PVP was even more profound as shown in Table 10. Again administration of 20 ml PVP alone had no effect.

TABLE 10
Rabbit with 20 ml PVP

| Agonist | Control | 15 min | 60 min | 24 hr | 48 hr | 72 hr |
|---|---|---|---|---|---|---|
| ADP | normal | 0% | 0% | 4% | 28% | 66% |
| Collagen | normal | 0% | 0% | 5% | 22% | 67% |
| Epinephrine | normal | 0% | 0% | 2% | 25% | 61% |
| Ristocetin | normal | 99% | 98% | 99% | 100% | 100% |

Not only is the platelet effect seen in vivo, there is some indication that the antibacterial effect is also present. Four rabbits were used for each dosage level. Three of the rabbits received grape-PVP and one received PVP alone as a control. Blood samples were taken prior to treatment and at various time points thereafter. When samples of plasma from a rabbit injected with 5 ml of grape-PVP were tested against MRSA following the protocol described above, it was found that the plasma showed an MIC titer of 1:32 and an MBC titer of 1:16. Plasma itself is known to have some antimicrobial properties. Control plasma showed an MIC titer of 1:16 and an MBC titer of 1:8. These experiments are shown with additional bacterial species below. Results are shown for pre-injection and 15 min, 1 hour and 24 hours post-injection. As expected, higher amounts of injected grape-PVP showed larger effects. Because uncontrolled intravascular coagulation is a dangerous side effect of sepsis, these results suggest that grape-PVP could be a useful treatment for bacterial blood infections because it controls platelet aggregation (and thus intravascular coagulation) as well as the bacterial infection.

TABLE 11
5 ml Dose of grape-PVP versus 5 ml Dose of PVP (MIC titer)

| Test Organism | Grape-PVP | PVP-alone |
|---|---|---|
| Listeria monocytogenes | | |
| Pre-injection | 1:4 | 1:4 |
| 15 min after | 1:8 | 1:8 |
| 1 hour after | 1:8 | 1:4 |
| 24 hour after | 1:4 | 1:4 |
| Pseudomonas. aeruginosa | | |
| Pre-injection | 1:4 | 1:4 |
| 15 min after | 1:16 | 1:4 |
| 1 hour after | 1:8 | 1:8 |
| 24 hour after | 1:4 | 1:4 |
| Yersinia enterocolitica | | |
| Pre-injection | 1:4 | 1:8 |
| 15 min after | 1:8 | 1:4 |
| 1 hour after | 1:8 | 1:4 |
| 24 hour after | 1:4 | 1:8 |
| Serratia marcescens | | |
| Pre-injection | 1:4 | 1:4 |
| 15 min after | 1:16 | 1:4 |
| 1 hour after | 1:8 | 1:8 |
| 24 hour after | 1:4 | 1:4 |
| Klebsiella pneumonia | | |
| Pre-injection | 1:4 | 1:4 |
| 15 min after | 1:16 | 1:4 |
| 1 hour after | 1:8 | 1:4 |
| 24 hour after | 1:4 | 1:4 |
| Staphylococcus epidermidis | | |
| Pre-injection | 1:4 | 1:4 |
| 15 min after | 1:8 | 1:4 |
| 1 hour after | 1:8 | 1:8 |
| 24 hour after | 1:4 | 1:4 |

TABLE 12
10 ml Dose of grape-PVP versus 10 ml Dose of PVP (MIC titer)

| Test Organism | Grape-PVP | PVP-alone |
|---|---|---|
| Listeria monocytogenes | | |
| Pre-injection | 1:4 | 1:4 |
| 15 min after | 1:32 | 1:4 |
| 1 hour after | 1:32 | 1:8 |
| 24 hour after | 1:8 | 1:4 |
| Pseudomonas. aeruginosa | | |
| Pre-injection | 1:4 | 1:4 |
| 15 min after | 1:16 | 1:8 |
| 1 hour after | 1:8 | 1:8 |
| 24 hour after | 1:4 | 1:4 |
| Yersinia enterocolitica | | |
| Pre-injection | 1:4 | 1:8 |
| 15 min after | 1:8 | 1:4 |
| 1 hour after | 1:8 | 1:8 |
| 24 hour after | 1:4 | 1:4 |
| Serratia marcescens | | |
| Pre-injection | 1:4 | 1:4 |
| 15 min after | 1:16 | 1:8 |
| 1 hour after | 1:8 | 1:4 |
| 24 hour after | 1:4 | 1:4 |
| Klebsiella pneumonia | | |
| Pre-injection | 1:2 | 1:2 |
| 15 min after | 1:16 | 1:2 |
| 1 hour after | 1:16 | 1:2 |
| 24 hour after | 1:2 | 1:4 |
| Staphylococcus epidermidis | | |
| Pre-injection | 1:4 | 1:4 |
| 15 min after | 1:32 | 1:4 |
| 1 hour after | 1:32 | 1:8 |
| 24 hour after | 1:8 | 1:4 |

TABLE 13
20 ml Dose of grape-PVP versus 20 ml Dose of PVP (MIC titer)

| Test Organism | Grape-PVP | PVP-alone |
|---|---|---|
| Listeria monocytogenes | | |
| Pre-injection | 1:4 | 1:4 |
| 15 min after | 1:64 | 1:8 |
| 1 hour after | 1:64 | 1:8 |
| 24 hour after | 1:8 | 1:4 |
| Pseudomonas. aeruginosa | | |
| Pre-injection | 1:4 | 1:4 |
| 15 min after | 1:16 | 1:8 |
| 1 hour after | 1:8 | 1:4 |
| 24 hour after | 1:4 | 1:4 |
| Yersinia enterocolitica | | |
| Pre-injection | 1:4 | 1:4 |
| 15 min after | 1:8 | 1:4 |
| 1 hour after | 1:8 | 1:8 |
| 24 hour after | 1:4 | 1:4 |
| Serratia marcescens | | |
| Pre-injection | 1:4 | 1:4 |
| 15 min after | 1:16 | 1:4 |
| 1 hour after | 1:8 | 1:4 |
| 24 hour after | 1:4 | 1:8 |
| Klebsiella pneumonia | | |
| Pre-injection | 1:2 | 1:2 |
| 15 min after | 1:64 | 1:2 |
| 1 hour after | 1:64 | 1:4 |
| 24 hour after | 1:8 | 1:2 |
| Staphylococcus epidermidis | | |
| Pre-injection | 1:4 | 1:4 |
| 15 min after | 1:64 | 1:8 |
| 1 hour after | 1:64 | 1:4 |
| 24 hour after | 1:32 | 1:4 |

The above results suggest that the antimicrobial effect of the grape-PVP is cleared more rapidly than the platelet effect. Because the platelets can be largely restored to control conditions by washing, this may mean that a significant amount of the antimicrobial material becomes bound and is no longer effective. Alternatively, the anti-platelet and the antimicrobial compounds may no be the same and the antimicrobial compounds might be metabolized or excreted more rapidly than the anti-platelet compounds.

For chronic control of platelet aggregation it is not preferred to administer a treatment by injection. It is known that most if not all of the grape-PVP components are absorbed when the material is injected. The excretion of the components can be readily measured in the patient's urine. It is anticipated that continual oral administration of grape-PVP will result in modulation of platelet aggregation.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

I claim:

1. A method of extending the life of isolated platelets comprising the step of adding polyphenolic-PVP extract to the isolated platelets.

2. The method according to claim 1, wherein the polyphenolic-PVP extract is grape-PVP.

3. The method according to claim 1 further comprising the step of washing the isolated platelets to remove the polyphenolic-PVP extract.

4. The use of polyphenolic-PVP extract in the manufacture of a medicament for inhibiting platelet aggregation in a mammal.

5. The use according to claim 4, wherein the polyphenolic-PVP extract is selected from the group consisting of Goji-PVP and grape-PVP.

6. The use of grape-PVP in the manufacture of a medicament for reducing the number of oral bacterial in a human.

7. The use according to claim 6, wherein the medicament comprises an oral rinse containing grape-PVP.

8. The use according to claim 6, wherein the medicament comprises a chewable composition containing grape-PVP.

9. The use of grape-PVP in the manufacture of a medicament for controlling infecting bacteria.

10. The use according to claim 9, wherein the medicament is a material for topical application.

11. The use according to claim 9, wherein the medicament is a material for intravenous injection.

12. The use according to claim 9, wherein the infecting bacteria are MRSA.
